(19) United States
(12) Patent Application Publication
(10) Pub. No.: US 2008/0249384 A1
(43) Pub. Date: Oct. 9, 2008

(54) System and Method for Estimating the Glucose Concentration in Blood

(75) Inventors: Ole Skyggebjerg, Hillerod (DK); Kristian Glejbol, Glostrup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO Nordisk A/S, Bagsvaerd (DK)

(21) Appl. No.: 11/661,867

(22) PCT Filed: Sep. 5, 2005
(86) PCT No.: PCT/EP2005/054360

§ 371 Date: Jan. 29, 2008

(30) Foreign Application Priority Data
Sep. 3, 2004 (DK) PA 2004 01333

(51) Int. Cl. A61B 5/055

(52) U.S. Cl. 600/347

(57) ABSTRACT

This application relates to glucose monitoring systems for continuously measuring the glucose concentration in a patients blood. The system is adapted to communicate with one or more sensors (10, 20) for transcutaneous insertion into a patient and for producing sensor signals related to the glucose concentration. The system comprises an electronic calculator unit and a display (14) for displaying the measured glucose concentration. The electronic calculator unit further comprises means for calculating an estimate of the uncertainty, i.e. the degree of accuracy of the glucose measurement, and the display is configured for displaying an interval representing said uncertainty (15).

### FIELD OF THE INVENTION

This invention relates to procedures for estimating the glucose concentration in blood using biosensors, in particular using transcutaneous electrochemical sensors suitable for in vivo measurement of metabolites.

### BACKGROUND OF THE INVENTION

In recent years, a variety of implantable sensors have been developed for in vivo measurements of various biological parameters. Among these transcutaneous sensors (ie sensors mounted through the skin) show promise for real-time measuring of important biological parameters like acidity of the blood and concentration of metabolites and blood gasses.

One of the most prominent examples of the use of implantable sensors is within the field of blood glucose (BG) measurements. BG information is of the utmost importance to diabetics, as these readings are instrumental in the adjustment of the treatment regimen.

The conventional way to obtain BG information is by applying minute amounts of blood to test strips. Although simple and reliable, this method gives only discrete readings and thus not a complete understanding of the BG at any time. A new development is transcutaneous sensors where the sensor is implanted under the skin. As the sensor is always in contact with biological fluids, this opens the possibility for continuous measurements. Continuous BG readings obtained with little or no delay will be particularly useful in numerous ways. First of all, the continuous monitoring will help preventing hypoglycaemic incidents and thus contribute to a vast increase in the quality of life of the diabetic patient.

Although the invention described in this application is not limited to systems for BG measurements, BG measurements will be used in the following text to exemplify all relevant aspects of the invention.

In general, readings from a transcutaneous sensor reflect only to some extent the value found in undisturbed tissue. An exact reading is not obtainable due to the metabolic changes in the tissue caused by the damage inflicted during insertion. The relation between readings in disturbed tissue and the actual value in undisturbed tissue is therefore unknown in the general case.

If transcutaneous sensors are used to indicate the concentration of species in the bloodstream, the relation between the reading and the actual value becomes even more complex due to time lag between the concentration found in the blood and the value read by the sensor. This is the case in particular for BG measurements, as BG sensors are most often implanted in the subcutaneous tissue although the value of interest is the concentration of glucose present in the bloodstream.

To summarize, the measured value of eg glucose found in the subcutaneous tissue reflects to some degree the concentration found in the bloodstream although a time lag between the reading and the actual value exists. For glucose the time-corrected concentration in the subcutaneous tissue is in general lower than in the bloodstream due to physiological factors as well as tissue damage. Thus the readings even from an ideal subcutaneous sensor will represent only the actual value found in the blood if corrected for the unknown proportionality factor as well as time-lag.

In patent application No. US 2002/0161288 A1 an approach to calibration is claimed that employs numerous calibration values taken at predetermined intervals. According to the method described in this patent, sampling has to be carried out at predetermined intervals until two consecutive calibration factors fall within a certain interval. Thereafter a readout of the measured glucose concentration can be presented on a display.

If follows firstly from the above circumstances that the prior art is vitiated by the drawback that—when a new sensor is to be started up—it is necessary to perform calibrations and then wait a while for it to be verified, by means of electronic circuits, that the deviation between the measurements/calibrations is sufficiently low. Secondly, the prior art is associated with the drawback that, for safety considerations, the users are “deprived” of the option of regulating themselves the accuracy of the readings because the accuracy is programmed into the electronic circuits. For instance, there may be situations where the user desires to accept a higher degree of uncertainty than average. This may be at the theatre, where it is inconvenient if an alarm is suddenly produced that might just as well have been postponed a couple of hours.

### OBJECT OF THE INVENTION

One object of the invention is to devise a novel method of collecting, processing and presenting data obtained in systems employing at least one biosensor, hereby increasing the flexibility and convenience of the system without reducing its safety and reliability.

This object is accomplished in that an estimate is provided of the uncertainty, i.e. the degree of accuracy of the glucose-concentration measurement, and that a result is displayed on a display comprising the display of an interval representing the estimated uncertainty.

By showing the user the uncertainty interval it is possible, on the one hand, to use the measurement entirely without preceding calibration and time-lag and, on the other, it is sound from a safety point of view to allow the user himself to adjust the accuracy in connection with eg the alarm functionality.

According to one further aspect of the invention, the display presents the readings as an interval of possible values rather than an exact number. Depending on the quality of the achieved data, a wider or narrower interval will be displayed.

The display is controlled by a microprocessor which, based on available data, calculates the interval within which the real measured value is to be found. By available data is meant data from the biosensor as well as calibration data from other devices and sensors.

One of the major advantages provided by the invention is that it is possible to obtain a readout of a measured blood-glucose value albeit the uncertainty is comparatively high. As it is, there may be situations in which the user will appreciate this option which does not involve any risk; the level of uncertainty being, as mentioned, displayed to the user. Another major advantage is that the uncertainty can be reduced considerably merely by one calibration, because the uncertainty is calculated on the basis of maximum and minimum values from the sensor that are observed during a predetermined period of time which is time-lagged in relation to the calibration measurement. By performing further valid calibration measurements, the uncertainty can be further reduced, which will become immediately apparent on the display. Thus, the user is able to perform precisely the number of calibration measurements it takes to achieve a desired narrowing of the uncertainty interval. Valid calibration measurements are intended to designate that such measurements are disregarded that exhibit obvious errors, or where the measurement is too old. If a number of calibrations are performed successively, the measurement accuracy will improve with time by the reciprocal of the square root of the number of measurements; however, this requires that the measurements are not too old. It is to be noted that the great advantages provided by the invention appears by no, one single or few calibrations for the mere reason that by performing many calibrations, also in accordance with the prior art, it is possible to accomplish a relatively small uncertainty; a scenario where it is of comparatively less interest to calculate and display the uncertainty interval.

As mentioned, the invention provides the advantage that, from a safety point of view, it is perfectly all right to display the result of a blood glucose measurement. This is due not only to the invention overcoming the prejudice that one cannot display an uncertain measurement value, but also to the circumstance that measurement sensors are increasingly improved, and thus it is exclusively the uncertainty of the tissue which is decisive at the beginning. This will typically give rise to an uncertainty between 0 and −30%. Irrespective of the safety, it is also a feature of the invention that, from the onset, it is possible to verify whether the glucose concentration is increasing or decreasing as soon as a brief initial operational period of the sensor has elapsed. Moreover, sensors are advantageously employed that are provided with calibration information to the effect that the sensor can be regarded as being essentially flawless.

As mentioned the uncertainty can be reduced by performing one or more calibration measurements, in which context it is also important to note that, in accordance with the invention, an interval is shown representing the uncertainty albeit it decreases as more calibrations are being performed. Owing to tissue changes, lower validity is ascribed to earlier calibration measurements compared to recent calibration measurements.

As mentioned above, the invention provides the advantage that it is sound from a safety point of view to enable the user to adjust the safety margin used on connection with an electronic circuit. The adjustments may also take place automatically in dependence on the uncertainty estimated in accordance with the invention. Measurement equipment and alarm circuit may be closely integrated functionality-wise, and the user himself may be enabled to program threshold values for the alarm circuit. It is well-known that uncertainty regarding low blood-glucose values is more serious than uncertainty regarding high blood-glucose values. Typically, the alarm circuit will be configured for taking this into account. It is also well-known that some situations are critical, while others are not—eg it is critical to drive a car, while it is not critical to rest.

The presentation on a display can be accomplished in a variety of ways by means of numbers, graphics, colours, sound signals; bearing in mind, though, that diabetics are often elderly and visually impaired.

The invention is also particularly suitable in connection with a calibration process of a sensor, while the previously used sensor is still active. The latter calibration technique will generally be able to reduce the calibration time; and when this feature is combined with the present invention, readouts will result that are even quicker and even more useful than previously.

The invention also relates to a system for calculating and displaying measurement results from a subcutaneous sensor. The system is characterized in that an electronic calculator unit is provided having means for producing an estimate of the uncertainty of the glucose measurement; and that the display is configured for displaying an interval representing the estimated uncertainty. The system may be provided as one self-contained apparatus. The apparatus may contain one or more sensor units or may be communicating with one or more sensor units. Alternatively, the electronic calculator unit may be physically divided from the means for generating an output on a display. Thus, the presentation of the output may be provided on a separate device communicating with the electronic calculator unit.

Since the user is now able to obtain information on the magnitude of the uncertainty of the measurement it is now possible—as will appear from the following—to obtain many considerable advantages. Therefore, it is also important to be able to display the interval as illustratively as possible; preferably by graphical representation. The users' perception of what is a clear and safe representation may differ widely, and therefore the apparatus is preferably configured such that the display is able to display the interval in different graphical ways that can be selected by the individual user.

In the preferred embodiment the sensor is directly coupled to an electronic circuit that is configured for being able to communicate with the electronic calculator unit. The assembly concerned may both be a disposable assembly where the sensor and said electronic circuit are built integrally and discarded as a whole when the longevity of the sensor is exceeded; or it may also be a disposable sensor connected to a multiple-use electronic circuit. According to a preferred embodiment the electronic circuit in the sensor contains calibration information that can typically be produced in the manufacture of a series of sensors.

Preferably the electronic calculator unit comprises a data storage for calibration information, which information can be accomplished in various ways and communicated to the data storage in various ways.

The apparatus according to the invention is able to provide not only the uncertainty of a first measurement without preceding calibration measurement—it can also be used for reducing the uncertainty on the measurements in that the electronic calculator unit is able to perform iterative calculations on the basis of the information available in the data storage. The information may be generated by the electronic calculator unit itself, or it may be generated by eg a test-strip glucose-measurement device. As a further alternative, the means for producing said information may comprise a further transcutaneous sensor that has been in operation for some time already.

Preferably said apparatus parts comprise transmitter and receiver circuits for wireless communication of said data/information.

According to a preferred embodiment, the electronic calculator unit also comprises an electronic alarm circuit; and the apparatus has means, such as push buttons, by means of which the user is able to adjust the threshold values of the alarm circuit. This feature which is of great significance to a user is now enabled in practice, because the user is able to set the alarm values on the basis of a displayed margin of uncertainty. So far the user has not been allowed to perform such changes as it would be far too risky when the measurement uncertainty is unknown.

The invention also puts an end to the prejudice that automatic or semi-automatic apparatuses for administering a medicament can be controlled only when the measurement uncertainty has been reduced to a pre-defined minimum. In particular in connection with semi-automatic dosage units, the user will be granted much more freedom in using the apparatus since display of the uncertainty interval may preclude the risks that have so far limited the applicability of the known apparatuses.

The invention will now be explained in further detail in the following specification of exemplary embodiments; reference being made to the drawing, wherein:

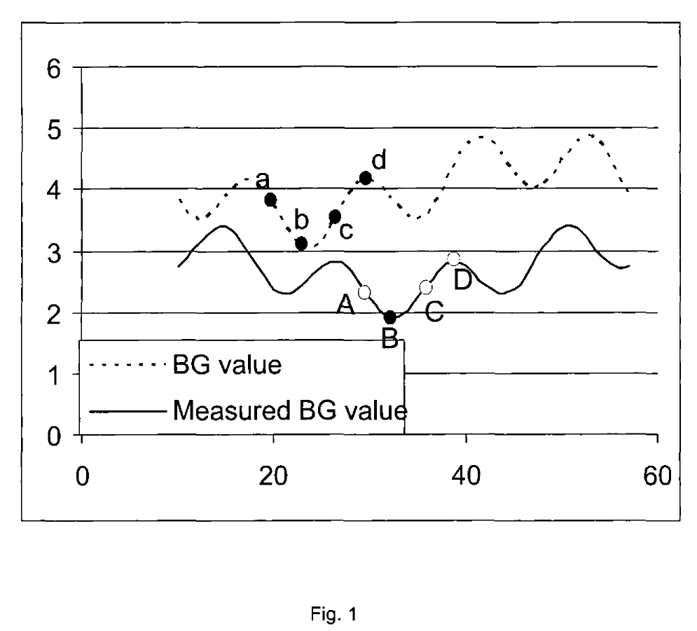

FIG. 1 shows relations between a true glucose concentration in the blood and a measured value from a sensor;

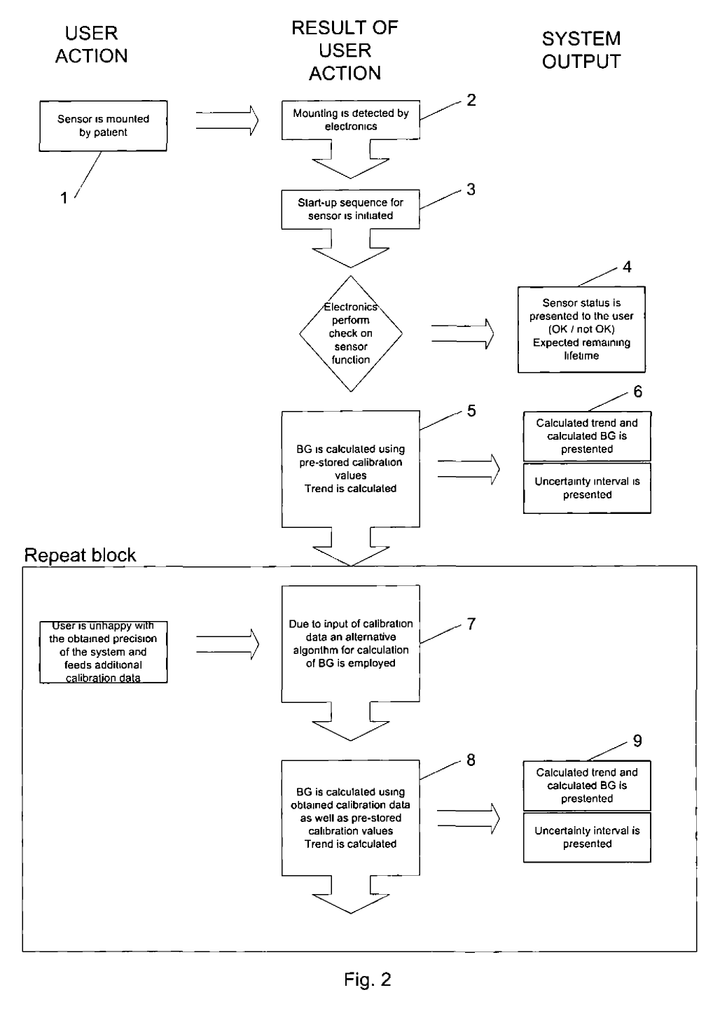

FIG. 2 shows a flow chart illustrating the method according to the invention and illustrates how it is possible to accomplish an estimate of the uncertainty of the measurements;

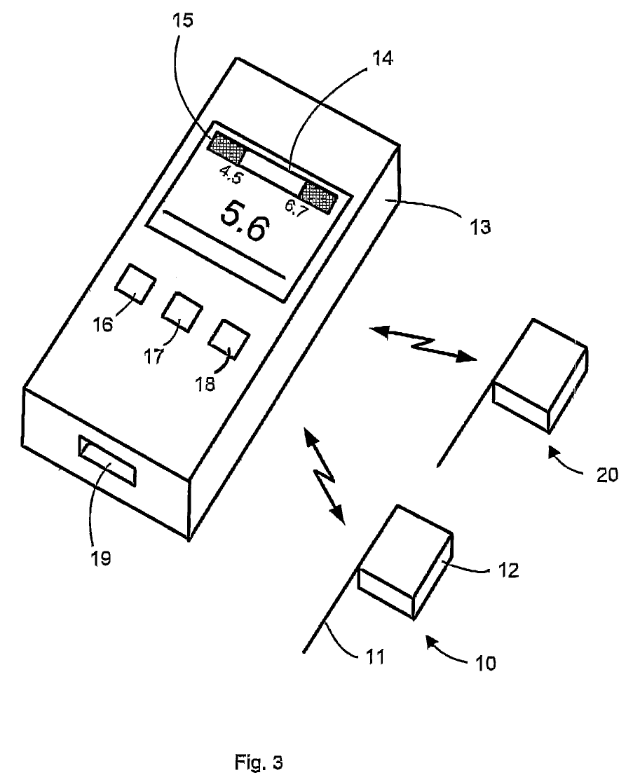

FIG. 3 illustrates an embodiment of the invention; while

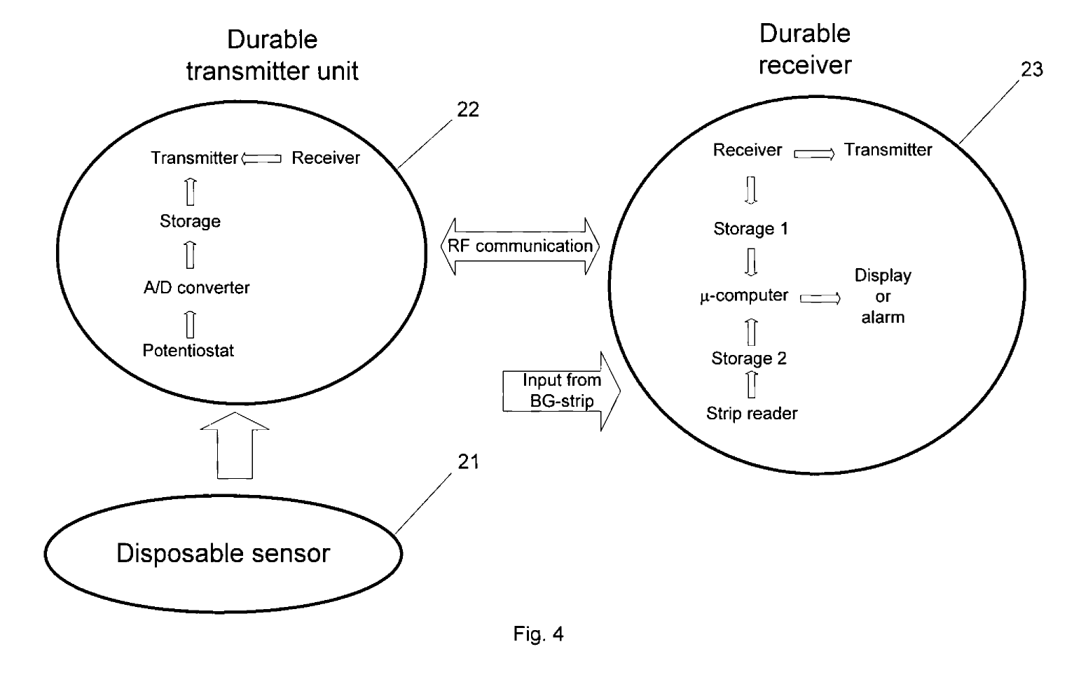

FIG. 4 illustrates the electronic functionality units that may partake in the apparatus, eg the one shown in FIG. 3.

### DETAILED PART OF THE DESCRIPTION

The principle of the invention will first be described with reference to FIG. 1 with the object of showing how, in practice, it is possible to calculate the uncertainty intervals presented to the user.

Especially for the algorithms calculating the calibration constants numerous approaches are valid, thus the example below is only depicting one possibility among many.

In the general case calibration values are weighted such that new values count more than older ones. This is not included in this example in order to keep the mathematical expressions simple.

Also the validity of older calibration measurements degrade with time. This is conveniently accomplished by letting ΔM grow with time. This is not included in this example in order to keep the mathematical expressions simple.

Definitions:

BG value=Real BG value, i.e. the value one would obtain from a perfect CGM system f(t)=The variation of BG value with time.

f(t) is not known. Only discrete points are known in the general case. These discrete points are found by strip measurements.

Measured BG value=The raw data coming from the CGM system.

F(t)=The recorded raw data

F(t) is known as data is continuously stored in the monitoring device

ΔM=Uncertainty on the acquired BG value due to handling and variations in strip production.

Ignoring uncertainties in the system the correlation below is given

f(t)=(F(t+δ)+Cₒ)Cₚ (eq 1)

Where

δ=Time lag. δ might either be calculated from the correlation between calibrations and F(t) values or δ might be a fixed pre-programmed value.

According to this example δ is pre-programmed by the user, based on experience. ε=The uncertainty of δ. If e.g. the user is in a hot bath the rate of blood flow through the outer capillaries is high and a typical time lag will be (δ−ε). If the user is outdoors and freezes, the outer capillaries of the skin will contract whereby the blood flow in the outer capillaries is reduced. In this situation a typical lag time is (δ+ε). ε is typically found during initial calibration of the system.

Cₒ=Offset value. Cₒ might either be calculated from the correlation between calibrations and F(t) values or Cₒ might be a fixed pre-programmed value.

According to this example, Cₒ is pre-programmed into a memory module mounted on the sensor.

Cₚ=Constant of proportionality. Cₚ might either be calculated from the correlation between calibrations and F(t) values or Cₚ might be a fixed pre-programmed value.

According to this example Cₚ is estimated until calibration data exist.

Cₚ=Cₚₚ*Cₚₛ where Cₚₚ is specific for person using the sensor whereas Cₚₛ is specific to the sensor. In this example information on Cₚₛ is pre-programmed into a memory module mounted on the sensor.

Including uncertainties equation (1) expands to

$$\begin{matrix}\frac{f ( t - δ ) - Δ M}{max ( F ( t - ɛ ) … F ( t + ɛ ) ) + {C}_{o}} \leq {C}_{p} \leq \frac{f ( t - δ ) + Δ M}{min ( F ( t - ɛ ) … F ( t + ɛ ) ) + {C}_{o}} \\ OR & ( eq 2 ) \\ \frac{f ( t - δ ) - Δ M}{( max ( F ( t - ɛ ) … F ( t + ɛ ) ) + {C}_{o} ) * {C}_{p s}} \leq {C}_{pp} \leq \frac{f ( t - δ ) + Δ M}{( min ( F ( t - ɛ ) … F ( t + ɛ ) ) + {C}_{o} ) * {C}_{p s}} & ( eq 2 a )\end{matrix}$$

If multiple calibration measurements exist the significance of ΔM decreases, thus yielding the expression

$${\sum }_{n} \frac{f ( t - δ ) - \frac{Δ M}{\sqrt{n}}}{n ( max ( F ( t - ɛ ) … F ( t + ɛ ) ) + {C}_{o} )} \leq {C}_{p} \leq {\sum }_{n} \frac{f ( t - δ ) + \frac{Δ M}{\sqrt{n}}}{n ( max ( F ( t - ɛ ) … F ( t + ɛ ) ) + {C}_{o} )} \qquad ( eq 3 )$$

Upon mounting the sensor assembly is activated. By measuring the current flowing through the sensor during start-up it will be possible to detect whether the sensor is mounted correctly. If the electronic circuits detect that the current rises in a correct manner an “OK” message is signaled to the monitoring device.

Upon full wetting of the sensor a voltage pulse is initiated conditioning the sensor for further service.

After initial pulse, the monitoring device communicates the interval within which the blood-glucose value is expected to be. For this calibration free value, eq (1) is used for calibration. Depending on the precision of the sensor and the tissue damage following the mount the values of Co and Cp varies. According to this example Co is exactly known prior to insertion whereas Cp is known ±30%. Although ±30% might be critical for low glucose concentrations this value is sufficient precise if the system measures the blood-glucose to be in the middle/upper part of the allowed range.

Of interest is furthermore that the variations in blood glucose can already now be detected, i.e. a drop/rise in BG level is without any further calibrations precisely detectable on a relative scale.

If the user wants better information on the blood-glucose, an additional calibration can be carried out, e.g. using a strip measurement as known in the art. The result of the strip measurement can be communicated to the monitor either automatically or manually.

Now the Cp value is recalculated according to the formula.

(Eq1+Eq2)/2

By using this formula the calibration factor Cp is compounded partly from a preset calibration value and partly from a calibration value obtained on the specific sensor.

If even improved precision is wanted, further calibrations can now be performed. For more than one calibration values, the formula Eq (3) is used. By using Eq (3), all data for calibration is obtained on the specific sensor of interest. Hence the best possible calibration possible is obtained.

Note that according to the invention, Cp will always be calculated as an interval.

FIG. 2 shows a flow chart for illustrating how a user is able to execute the method according to the invention. At 1 the user positions a new sensor subcutaneously, following which the electronic circuits detect whether the sensor is positioned correctly, see 2 in FIG. 2. When the sensor is positioned correctly, it will emit signals that the circuits are able to detect and take as an expression of correct positioning. At 3, a further start-up action is outlined: ia comprising an over-voltage impulse to the sensor. When the sensor is fully wefted, it is ready to perform reliable measurements within the uncertainty intervals explained above in connection with FIG. 1. Preferably the display shows a message to the user, see 4 in FIG. 2, following which the user knows that the apparatus is ready for use. The apparatus immediately starts to calculate the blood glucose concentration at 5 and to perform a first estimation of the uncertainty of the measurement. The estimation is based on calculations of the kind that will appear from equation 1 above, and at 6 the result of both the measurement and the found uncertainty interval is displayed. Preferably the trend of the measurement is also shown immediately as a function of time since, after function No. 4, the sensor provides absolutely reliable, relative measurements. The uncertainty concerns the absolute measurement and is displayed as an interval.

For certain uses measurements, albeit associated with an uncertainty of ±30%—see above, are valuable to the user, in particular in connection with relatively high glucose concentrations that are not life-threatening to the user. Here, great benefits can be obtained by being able to influence alarm functions, as will be explained further below. Some users are happy with relatively uncertain measurement values, while other users are very careful to obtain accurate measurement values, which require calibrations of the apparatus as explained below in connection with FIG. 2 (repeat block).

By a single traverse of repeat block, the function 7 enables a calculation in accordance with equation 2 above. That calculation depends exclusively on the continuous measurements performed by means of the sensor, and according to equation 2 the maximum and minimum values are used that are found within a predetermined period of time as long as a higher degree of accuracy has not been obtained for the time-lag that exists between a calibration measurement on the blood, eg by means of a strip measurement, and the corresponding glucose concentration in the tissue. Then, at 8, a new blood-glucose concentration is calculated as well as a new uncertainty interval that is displayed to the user on the display as shown by 9. If the user desires even less uncertainty on the measurement, the repeat block can be traversed more times following preceding strip calibration measurement and using the more advanced formulae given above.

It is noted that by performing many repeated calibration measurements, an information level is reached that does not differ substantially from that obtained by the prior art. The great advantages of the invention reside in the rather more flexible applications quite briefly after the taking into use of a new sensor due to the user being acquainted with the uncertainty associated with the measurements.

FIG. 3 shows an example of a concrete embodiment of A system according to the invention. The apparatus comprises a sensor 10 comprising an electrode 11 and an electronic circuit 12, preferably configured for being able to transmit measurement data to a portable unit 13. The sensor 10 may be of the type where the electrode is connected to multiple-use electronics, or it may be of the disposable assembly type, where both electrode and electronic circuits is one assembled unit that is discarded after use.

The portable unit 13 is configured for calculating and displaying values for the blood-glucose concentration that is measured by means of the electrode 11. The information is shown in a display 14 showing, in the shown embodiment, on the one hand the measured value (5, 6), and being—on the other and in accordance with the invention—a graphical representation of the uncertainty associated with the measurement. The graphical representation is shown in the field 15 that comprises a light field between the values 4, 5 and 6, 7. Outside these values the fields are dark, and the value 5, 6 is indicated by an arrow. In this manner the user is able to clearly read the measured value and have a clear impression of how uncertain, or rather how certain the measurement is. Uses are perceivable, where measurements with an uncertainty as high as ±30% are acceptable and informative, and measurements are perceivable where it is necessary to operate with a far smaller uncertainty. Representation of the uncertainty as an interval enables the user to very readily form an impression of what the measurement in question can be used for.

One of the uses is for the alarm circuits that are typically integrated in the unit 13 to the effect that an alarm, be it visible and/or audible, is emitted if the blood-glucose concentration becomes too low or too high. According to the invention the unit 13 comprises control buttons 16, 17 by means of which the user is able to influence the threshold values with which the integral alarm circuit operates. The options can be made considerably more flexible than has been possible so far, because the user may rely on the displayed interval of the uncertainty of the measurements. 18 symbolizes a push button by which it is possible to switch between several types of graphical representations of the uncertainty interval, thereby enabling the user himself to select the kind of display he/she feels most comfortable with.

19 represents an introduction opening for a strip, as the unit 13 may comprise an integral strip reader for measuring the glucose concentration in a drop of blood on the strip. Such measurement is used for performing calibrations of the unit 13. Alternatively it is an option to use a separate strip measurement device that may be in wireless communication with the portable unit 13. It is also an option that the portable unit 13 is able to communicate wirelessly with a further sensor 20.

FIG. 4 shows which functions are contained in a preferred embodiment of the apparatus according to the invention.

As will appear from FIG. 4, an example is selected where a disposable sensor 21 is used that is coupled to a durable transmitter unit 22. The transmitter unit 22 contains pre-amplifier circuits and A/D converters and a storage for storing measured values and optionally values received from the durable receiver 23. Preferably a disposable sensor is used, being in connection with its manufacture provided with information on a calibration factor, ie the conversion value between a measured sensor current and an associated blood-glucose concentration value. This information is also transmitted to the storage in the durable transmitter unit. In this manner the durable receiver 23 can start out by assuming that the uncertainty interval concerns exclusively the physical conditions (see C₀ in the explanation given in the context of FIG. 1). As shown in FIG. 3, the durable transmitter unit and the durable receiver are preferably configured for wireless communication and preferably the durable receiver 23 also contains a strip reader, thereby enabling transfer to the storage 2 of calibration values for the blood glucose concentration. The storage 1 is configured for being able to contain other information on calibration parameters or historical data that can be used for calculating a glucose concentration value and an associated interval that represents the uncertainty of the glucose concentration measurement. As outlined in the durable receiver 23, the micro-computer may be configured for performing the calculation processes explained above in the context of FIGS. 1 and 2.

1. A system for measuring glucose concentration comprising a transcutaneous sensor, an electronic calculator unit and a display for displaying the measured glucose concentration, characterized in that the electronic calculator unit has means for calculating an estimate of the uncertainty of the glucose measurement; and that the display is configured for displaying an interval representing said uncertainty.

2. A system according to claim 1 characterized in that the display is configured for graphical representation of the interval.

3. A system according to claim 2, characterized in that the display is configured for being able to produce various types of graphical representations of the interval.

4. A system according to claim 3, characterized in that the sensor comprises an electronic circuit configured for being able to communicate with the electronic calculator unit.

5. A system according to claim 4, characterized in that the electronic circuit in the sensor contains calibration information.

6. A system according to claim 5, characterized in that the electronic calculator unit comprises data storage for calibration information.

7. A system according to claim 6, characterized in that the data storage is configured for receiving information used in the electronic calculator unit for iteratively estimating the uncertainty of the blood-glucose measurement.

8. A system according to claim 7, characterized in that the apparatus has means for generating said data/information.

9. A system according to claim 8, characterized in that said means comprise a test-strip glucose-measurement device.

10. A system according to claim 8, characterized in that said means comprise a further transcutaneous sensor.

11. A system according to claim 1, characterized in that the apparatus comprises transmitter and receiver circuits for wireless communication of said data/information between the separate parts of the apparatus.

12. A system according to claim 1, characterized in that the apparatus comprises an electronic alarm circuit; and that the apparatus comprises means by which a user is able to adjust the threshold values of the alarm circuit.

13. A system according to claims 1, characterized in that the apparatus is configured for being able to communicate with a unit intended for dosing a medicament.

14. A system according to claim 1, characterized in that it comprises means for dosing and administering a medicament.

15. A method of estimating the glucose concentration in blood by means of a sensor inserted subcutaneously in live tissue and by means of associated electronic calculator circuits, characterized in that an estimate is provided of the uncertainty of the glucose-concentration measurement; and that a result is displayed on a display comprising the display of an interval that represents the estimated uncertainty.

16. A method according to claim 15, characterized in that the uncertainty is reduced by performing valid calibration measurements.

17. A method according to claim 16, characterized in that the uncertainty is estimated in response to the number of calibration measurements performed.

18. A method according to claim 16, characterized in that the number of calibration measurements is zero; and that the uncertainty is estimated in response to the signals emitted by the sensor during a pre-defined period of time.

19. A method according to claim 16, characterized in that the number of the number of calibration measurements is zero; and that the uncertainty is estimated on the basis of measured data and calibration parameters that are present either in the sensor and/or in connection with the calculator circuit.

20. A method according to claim 16, characterized in that at least one calibration measurement is performed; and that the uncertainty is calculated on the basis of maximum and minimum values from the sensor that are observed during a predetermined period of time which is time-lagged in relation to the calibration measurement.

21. A method according to claim 16, characterized in that the number of calibration measurements is larger than zero; and that the uncertainty is calculated on the basis of measured data, calibration measurements and the calibration parameters that are present either in the sensor and/or in connection with the calculator circuit.

22. A method according to claim 16, characterized in that the number of calibration measurements is larger than zero; and that the validity of earlier calibration measurements are reduced compared to more recent calibration measurements.

23. A method according to claim 15, characterized in that a signal is produced that represents said uncertainty; and that the signal is used as input signal for an electronic alarm circuit.

24. A method according to claim 23, characterized in that signals are produced that represent the calculated blood-glucose concentration and its estimated uncertainty; and that the signals are used as input signal for the alarm circuit.

25. A method according to claim 23, characterized in that the threshold values of the alarm circuit is pre-programmed by a user.

26. A method according to claim 23, characterized in that the alarm circuit is controlled to act differently in dependence of whether the upper or the lower threshold value is exceeded.

27. A method according to claim 26, characterized in that the information regarding the uncertainty is weighted such that the values are regarded to be more critical in case of relatively low blood-glucose concentrations than for relatively high blood-glucose concentrations.

28. A method according to claim 23, characterized in that the threshold values of the alarm circuit are changed in response to the in-use situation.

29. A method according to claim 23, characterized in that the threshold values of the alarm circuit are shown on said display.

30. A method according to claim 15, characterized in that the display comprises the size of the estimated uncertainty.

31. A method according to claim 15, characterized in that the display represents the blood-glucose value and the size of the uncertainty.

32. A method according to claim 15, characterized in that the display comprises the highest and lowest possible values of the blood glucose.

33. A method according to claim 15, characterized in that the display comprises numerical values.

34. A method according to claim 18, characterized in that the display comprises a graphical representation.

35. A method according to claim 15, characterized in that the display may both be graphical and numerical or a combination thereof.

36. A method according to claim 15, characterized in that, for the calculation of the information, signals are used from a further sensor that was introduced subcutaneously and emitted signals to said electronic calculator circuit for a period of time preceding the use of said sensor.
